(19) United States
(12) Patent Application Publication
(10) Pub. No.: US 2018/0333162 A1
(43) Pub. Date: Nov. 22, 2018

(54) SUBINTIMAL ENTRY CATHETERS AND METHODS FOR OCCLUSION CROSSING

(71) Applicant: SLEC LLC, Ada, MI (US)

(72) Inventor: Fadi Saab, Ada, MI (US)

(21) Appl. No.: 15/984,134

(22) Filed: May 18, 2018

Related U.S. Application Data
(60) Provisional application No. 62/508,961, filed on May 19, 2017.

(51) Int. Cl. A61B 17/22; A61F 2/95; A61B 34/00; A61M 25/00; A61M 25/01

(52) U.S. Cl. CPC A61B 17/22; A61F 2/95; A61B 34/73; A61M 25/007; A61M 25/0108; A61M 25/0194; A61B 2090/3966; A61B 2017/22071; A61B 2017/22052; A61B 2017/22074; A61B 2017/22001; A61B 2034/731; A61B 2017/22095

(57) ABSTRACT

A subintimal entry catheter can be used for crossing an occlusion in a blood vessel of a patient. The catheter can include a distal end portion and a lumen extending longitudinally within the catheter and radially to an opening in a first radial side at the distal end portion of the catheter. The catheter can be used to cross at least a portion of an occlusion through a subintimal space. A wire can be provided through the lumen and to the opening to reenter the true lumen of the blood vessel.

### CROSS-REFERENCE TO RELATED APPLICATIONS

This application claims the benefit of U.S. Provisional Application No. 62/508,961, entitled “SUBINTIMAL ENTRY CATHETERS AND METHODS FOR OCCLUSION CROSSING,” filed May 19, 2017, the entirety of which is incorporated herein by reference.

### FIELD

The present disclosure relates to treatment of stenosis in a body vessel and in particular to catheters and methods for crossing a chronic total occlusion in a blood vessel.

### BACKGROUND

Coronary artery disease (“CAD”) is the leading cause of death in the United States. One method for treating atherosclerosis and other forms of arterial lumen narrowing is percutaneous transluminal angioplasty, commonly referred to as “angioplasty” or “PTA,” or “PTCA” when performed in the coronary arteries. The objective in angioplasty is to restore adequate blood flow through the affected artery, which may be accomplished by inflating a balloon of a balloon catheter within the narrowed lumen of the artery to dilate the vessel. Similarly, Peripheral vascular disease (“PVD”) is an epidemic that is impacting millions of patients worldwide. The number of patients suffering from PVD is expected to increase by 15% in the western countries and 30% in the developing countries. Endovascular revascularization of patients with CAD and PVD is becoming a common modality of treatment in these patients.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 illustrates a plan view of a catheter system, according to some embodiments of the present disclosure.

FIG. 2 illustrates a side view of a catheter with ridges, according to some embodiments of the present disclosure.

FIG. 3 illustrates a sectional view of a distal end portion having a lumen, according to some embodiments of the present disclosure.

FIG. 4 illustrates a sectional view of a distal end portion having a tapered lumen, according to some embodiments of the present disclosure.

FIG. 5 illustrates a side view of a distal end portion having markers, according to some embodiments of the present disclosure.

FIG. 6 illustrates a side view of a distal end portion having an inflatable balloon, according to some embodiments of the present disclosure.

FIG. 7 illustrates a view of a catheter adjacent to an occlusion, according to some embodiments of the present disclosure.

FIG. 8 illustrates a view of the catheter of FIG. 7 crossing the occlusion and into a subintimal space, according to some embodiments of the present disclosure.

FIG. 9 illustrates a view of the catheter of FIG. 7 crossing past the occlusion and within a subintimal space, according to some embodiments of the present disclosure.

FIG. 10 illustrates an enlarged view of a distal end portion of the catheter of FIG. 7, according to some embodiments of the present disclosure.

FIG. 11 illustrates an enlarged view of a distal end portion of the catheter of FIG. 7 with a wire extending out of a lumen of the catheter, according to some embodiments of the present disclosure.

FIG. 12 illustrates an enlarged view of a distal end portion of the catheter of FIG. 7 with a balloon thereof in an inflated state, according to some embodiments of the present disclosure.

FIG. 13 illustrates an enlarged view of a distal end portion of the catheter of FIG. 7 with the wire extending into the lumen of the vessel, according to some embodiments of the present disclosure.

FIG. 14 illustrates a view of the catheter of FIG. 7 with the wire extending into the lumen of the vessel, according to some embodiments of the present disclosure.

FIG. 15 illustrates a view of the wire of FIG. 14 in the vessel after the catheter is withdrawn, according to some embodiments of the present disclosure.

FIG. 16 illustrates a view of an inflatable device deployed across the wire of FIG. 15, according to some embodiments of the present disclosure.

FIG. 17 illustrates a view of a stent deployed across the wire of FIG. 15, according to some embodiments of the present disclosure.

FIG. 18 illustrates an enlarged view of a pair of catheters within a subintimal space with a wire extending there between, according to some embodiments of the present disclosure.

FIG. 19 illustrates a view of a pair of catheters within a subintimal space with a wire extending there between, according to some embodiments of the present disclosure.

FIG. 20 illustrates a view of the wire of FIG. 19 in the vessel after the catheters are withdrawn, according to some embodiments of the present disclosure.

FIG. 21 illustrates a side view of a pair of catheters, according to some embodiments of the present disclosure.

FIG. 22 illustrates a side view of the catheters of FIG. 21 in a coupled arrangement, according to some embodiments of the present disclosure.

FIG. 23 illustrates a sectional view of a catheter of FIG. 21, according to some embodiments of the present disclosure.

FIG. 24 illustrates a sectional view of a catheter of FIG. 21, according to some embodiments of the present disclosure.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

Chronic total occlusion (“CTO”) is a term that describes an occlusion of the arterial conduit. The penetration of an occlusion in arteries or veins depends on the use of catheters and wires. A wire has to be across the occlusion to deliver revascularization therapy. The wire has to be directed from a patent segment of the vessel before the occlusion to another patent segment of the vessel after the occlusion. One challenge is to direct the wire from true lumen to true lumen. Commonly, all wires and traditional catheters will be directed into the subintimal plane. This area is not part of the true vessel and the physician has to direct the catheter with the wire to the true lumen. A common scenario is the wire leaving the vessel and becoming extra vascular. Long CTO's tend to be difficult to cross. The biggest obstacle to CTO crossing is subintimal position of any crossing device. The operator must enter into the true lumen of the vessel. One challenge is guiding a wire into the true lumen of the vessel. Most CTO crossing devices and wires/loop technique creates a large subintimal plane or become extra luminal making re-entry into the true lumen a difficult task.

According to some embodiments, a subintimal entry catheter can be used for crossing an occlusion. A treatment system 10, such as the one shown in FIG. 1, can be employed. The treatment system 10 shown in FIG. 1 can include a handle 20, a catheter 40 coupled with the handle 20, and a distal end portion 90 defining a region at or near a distal end of the catheter 40. The distal end portion 90 can include features for crossing an occlusion, entering a subintimal space, and delivering a wire or other components to the occlusion.

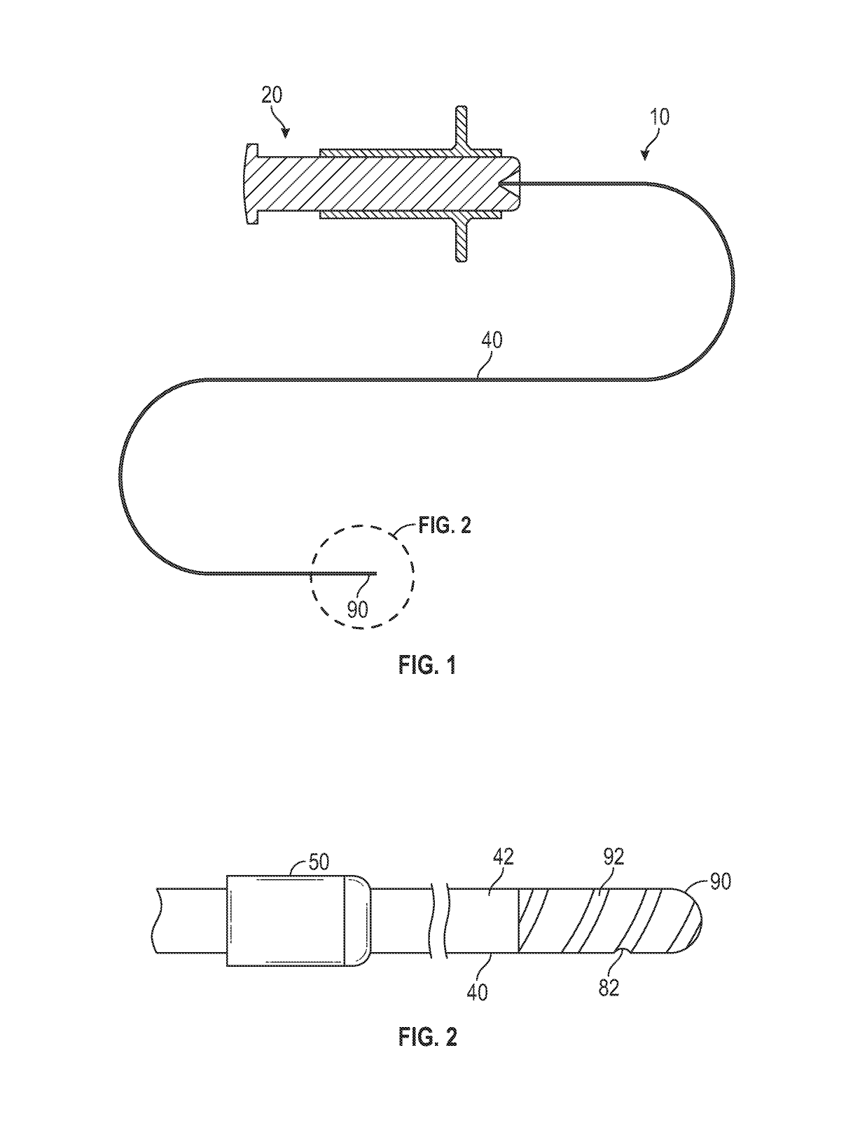

A catheter 40 can be used for crossing a occlusion. As shown in FIG. 2, the catheter 40 can have a low profile (e.g., about 0.018″ or less) and be supported by a guide catheter (e.g., 0.035″ or less). The guide catheter (not shown) can act as a protective shield, allowing better torque capabilities of the catheter 40. The catheter 40 can include a body portion 42 that is proximal to the distal end portion 90. The body portion 42 of the catheter 40 can include a braided core. The body portion 42 of the catheter 40 can further include a hydrophilic-coated material, to facilitate movement past the occlusion and tissue in the vessel. The distal end portion 90 can be more rigid that the body portion 42, so that the distal end portion 90 facilitates entry by penetration, and the body portion 42 facilitates a degree of bending to conform to anatomy.

In use, an operator can use the guide catheter to position the catheter 40 in a patient's vasculature. This procedure can involve inserting the guide catheter into the patient's vasculature through an access point such as the groin, and directing the distal portion of the guide catheter through the vascular system (e.g., with a guide wire) until it reaches the target vessel. After removing a guide wire from the guide catheter, the catheter 40 can be inserted into the guide catheter. As the distal end portion 90 of the catheter 40 exits the guide catheter, it can be positioned near the target site, such as an occlusion.

The catheter 40 can include features, such as a distal end portion 90 to act on the occlusion. For example, the distal end portion 90 can be rigid and shaped to facilitate entry. For example, the distal end portion 90 can be more rigid than the more proximal body of the catheter 40. The distal end portion 90 can be metal and tapered (e.g., conical or hemispherical shaped). Additionally or alternatively, the guide catheter can have ridges 92 and/or protrusions that extend helically along an outer surface thereof, to facilitate axial movement relative to a occlusion based on rotational action of the guide catheter. The ridges 92 and/or protrusions can be created in an angulated or horizontal fashion. By rotating the catheter 40 (e.g., with a torque device 50), the distal end portion 90 can be propelled through the occlusion or other materials, including tissue. The low profile nature of the catheter 40 can help it stay within the subintimal space. In case the device crosses into a subintimal plane, the catheter 40 can maintain proximity to the true lumen of the vessel. The ridges 92 can extend in a clockwise and/or counter-clockwise orientation. Variation in the angle of ridges is contemplated. One or more of the ridges 92 can be parallel to each other. The function of the ridges 92 can facilitate plaque penetration while controlling propulsion of the device. Additionally or alternatively, the distal end portion 90 can include a smooth surface. Additionally or alternatively, the distal end portion 90 can include a rough surface.

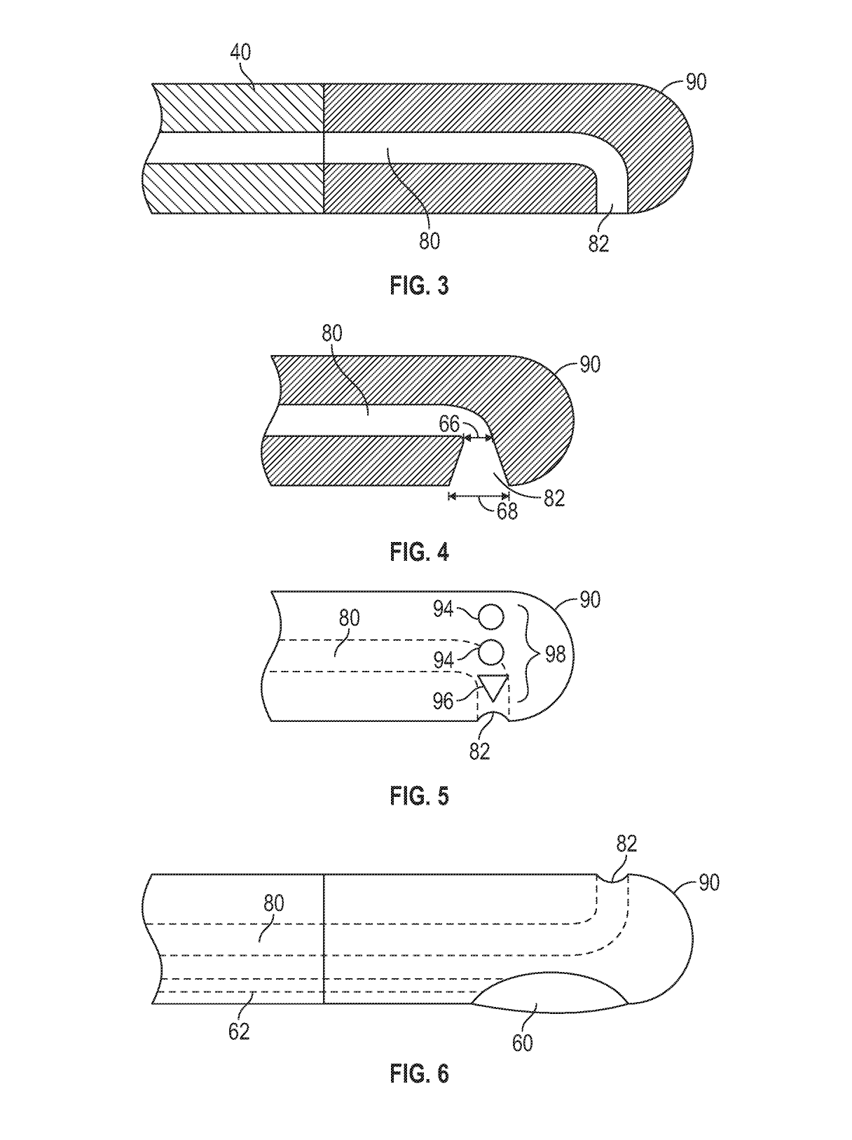

As shown in FIG. 3, the distal end portion 90 can include a lumen 80 extending within a length thereof. The lumen 80 can extend longitudinally along a region of the catheter 40 and/or the distal end portion 90 and connecting to the handle. At the distal end portion 90, the lumen 80 can extend to an opening 82 at a radial side of the distal end portion 90. A transition to the opening 82 can facilitate guidance of a wire through the lumen 80 to the opening 82. The opening 82 has features for connecting the outside of the catheter 40 to the lumen 80 of the catheter 40. At the opening 82, the lumen 80 can extend in a direction that is transverse (e.g., orthogonal) to the longitudinal (e.g., central) axis of the catheter 40 and the distal end portion 90. By rotating the catheter 40, the opening 82 can be positioned against tissue adjacent to a side of the distal end portion 90.

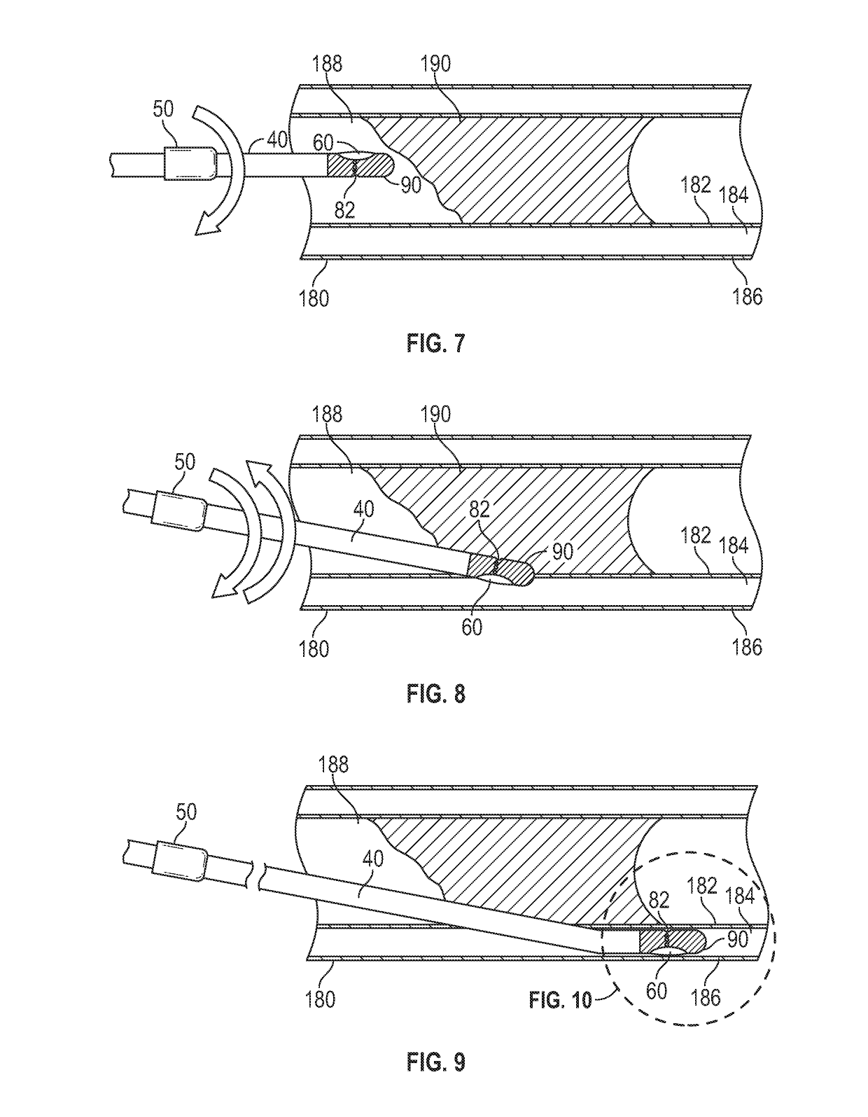

As shown in FIG. 4, the opening 82 can have a variable cross-sectional size and/or shape. The lumen 80 and the opening 82 allows passage of a wire (e.g., 0.014-0.018″ wire). The opening 82 can provide an outer portion that is larger than an inner portion of the lumen 80, thereby making it easier for wires to enter from the outside to the inside lumen 80 of the catheter. The cross-sectional size can taper or otherwise vary from an inner portion 66 (e.g., 0.005-0.025″, for example about 0.014″) to an outer portion 68 (e.g., 0.025-0.040″, for example about 0.035″). The angle created from the boundaries of the lumen 80 from the inner portion 66 to the outer portion 68 can range from 15-90 degrees. As shown in FIG. 7, the passage from the inner portion 66 to the outer portion 68 can have a conical and/or frusticonical shape. Other shapes are also contemplated.

As shown in FIG. 5, the distal end portion 90 can include one or more markers 98 to facilitate orientation of the distal end portion 90. Once the catheter is advanced in the vessel beyond the occlusion, the distal end portion 90 can be rotated with the opening 82 toward the true lumen of the vessel. Multiple (e.g., three or more) radiopaque markers 98 can be included. The markers 98 can have different shapes and/or sizes to distinguish them from each other. For example, one or more markers 94 can be one shape (e.g., circles), and one or more markers 96 can be another shape (e.g., triangle). Other shapes (e.g., square, rectangle, polygonal, oval, arrow, etc.) are contemplated. The marker 96 that is closest to the opening 82 can have a directional feature (e.g., triangle or arrow) in the direction of the opening 82. For example, as shown in FIG. 5, the tip of the triangle points toward the opening 82. The operator can change the orientation of the distal end portion 90 to make sure that the shapes are lined up and not overlapping, with the tip of the triangle pointing to the true lumen. This can allow the operator to advance the wire from inside the catheter to the true lumen of the vessel, as discussed further herein.

Once the side is directed toward the true lumen, a wire can be advanced and penetrate the subintimal plane to the true lumen. At this stage, the catheter can follow the wire, thus achieving the objective of having a wire connecting the true lumen of the vessel to the true lumen of the vessel on opposite sides of the occlusion.

As shown in FIG. 6, the distal end portion 90 can include an expansion member on a radial side of thereof. For example, an inflatable balloon 60 can be provided on radial side of the distal end portion 90. The balloon 60 can be positioned radially opposite the opening 82. For example, rather than extending about an entire circumference of the distal end portion 90, the entire balloon 60 can be on a radial side (e.g., of a central axis) that is opposite another radial side (e.g., of the central axis) containing the opening 82. Additionally or alternatively, multiple balloons 60 can be provided, with at least one be positioned at least on a side of the distal end portion 90 that is radially opposite the opening 82. The balloons can be separated from the opening and a wire therein by rigid (e.g., metal) body of the distal end portion 90, such that deflection by the balloon acts on the rigid body of the distal end portion 90 to move the rigid body in a direction away from the balloon 60. Expansion of some or all of the balloon 60 can be in a direction that is away from the opening 82.

In use, while the balloon 60 is flat (e.g., deflated), it will not impact advancing the catheter with the distal end portion 90. This can allow the distal end portion 90 to be advanced with a guide catheter. The balloon 60 inflation profile can allow for eccentric deflection. When inflated, the balloon 60 can expand to the side of the distal end portion 90. This can allow the distal end portion 90 to be pushed toward the true lumen of the vessel. Once the opening 82 is oriented toward the true lumen of the vessel, the operator may inflate the balloon 60 with an inflation medium (e.g., fluid) provided from the handle via an inflation lumen in fluid communication with the balloon 60. Inflating the balloon can allow the distal end portion 90 to be pushed closer to the true lumen of the vessel. If the opening 82 is closer to the true lumen of the vessel, advancing a wire into the true lumen can be easier. For example, the distance between the subintimal plane and the true lumen becomes smaller, and the wire advanced from inside the catheter to the true lumen of the vessel can travel a shorter distance.

The expansion member can include other or additional mechanisms. For example, a self-expanding spring (e.g., Nitinol) or other elastic structure can be provided opposite to the opening 82. The elastic structure can be initially sheathed in a compressed state. The elastic structure can be unsheathed with a release mechanism from the handle. Once released, it can allow the distal end portion 90 to be pushed in a direction away from the direction in which it is facing. In use, the operator can point the opening 82 toward the lumen of the vessel. Once the elastic structure is released, it can push the distal end portion 90 further toward the lumen of the vessel. The release mechanism located at the proximal end of the catheter can be a sliding cover that is controlled from the handle. Once pulled back, it can expose the elastic structure. If desired, it can advance over and collapse the elastic structure

Additionally or alternatively, features of the catheter 40 can be applied to another catheter (e.g., guide catheter) containing the catheter 40. For example, the expansion members can be incorporated onto the catheter 40 and/or a guide catheter containing the catheter 40. Deflection of the catheter 40 can be achieved, at least in part, by deflecting a guide catheter containing the catheter 40. By further example, markers can be incorporated onto the catheter 40 and/or a guide catheter containing the catheter 40. Orientation of the catheter 40 can be achieved, at least in part, by arranging the guide catheter in a desired orientation.

It may be undesired, difficult or impossible to pass directly through a occlusion in a lumen of a blood vessel to recanalize the vessel. In such instances, it may be possible to recanalize the blood vessel through a subintimal approach using the catheter 40. Turning to FIGS. 7-20, several aspects of an exemplary method for recanalizing an occluded blood vessel using the catheter 40 are illustrated.

The blood vessel 180 typically has three tissue layers, an innermost layer or intima layer (i.e., tunica intima) 182, an intermediate layer or media layer (i.e., tunica media) 184, and an outermost layer or adventitia layer (tunica adventitia) 186, with the media layer 184 positioned between the intima layer 182 and the adventitia layer 186. The intima layer 182 is a layer of endothelial cells lining the lumen 188 of the vessel 180, as well as a subendothelial layer made up of mostly loose connective tissue. The media layer 184 is a muscular layer formed primarily of circumferentially arranged smooth muscle cells. The adventitia layer 186, which forms the exterior layer of the vessel wall 180 is formed primarily of loose connective tissue made up of fibroblasts and associated collagen fibers. As will be described further herein, the distal end portion 90 can be advanced into a subintimal space (i.e., a space between the intima layer 182 and the adventitia layer 186) created in the vessel wall 180, such as through dissection of the tissue layers of the vessel wall 180.

As shown in FIG. 7, the catheter 40 can initially be advanced through the lumen 188 of the vessel 180 to a location proximate a proximal end of an occlusion 190 blocking the lumen 188. As shown in FIG. 8, the distal end portion 90 can then be advanced to penetrate outward through the intima layer 182 at a location proximal of the proximal end of the occlusion 190 into the vessel wall 180. For example, once the distal end portion 90 is in near or in contact with the cap of the occlusion 190, the operator can rotate the catheter 40. Ridges on the distal end portion 90 can engage with the occlusion 190.

As shown in FIG. 9, with the distal end portion 90 located between the intima layer 182 and the adventitia layer 186, the distal end portion 90 may be further advanced distally in a subintimal manner to create a subintimal space between the intima layer 182 and the adventitia layer 186. For example, the catheter 40 can be advanced in a subintimal manner until the distal end portion 90 is located distal of the distal end of the occlusion 190 in the subintimal space created, such as by dissection of the tissue layers of the vessel wall 180. It will be understood that the advancements of the distal end portion 90 can be facilitated by a guidewire and/or guide catheter preceding advancement of the distal end portion 90.

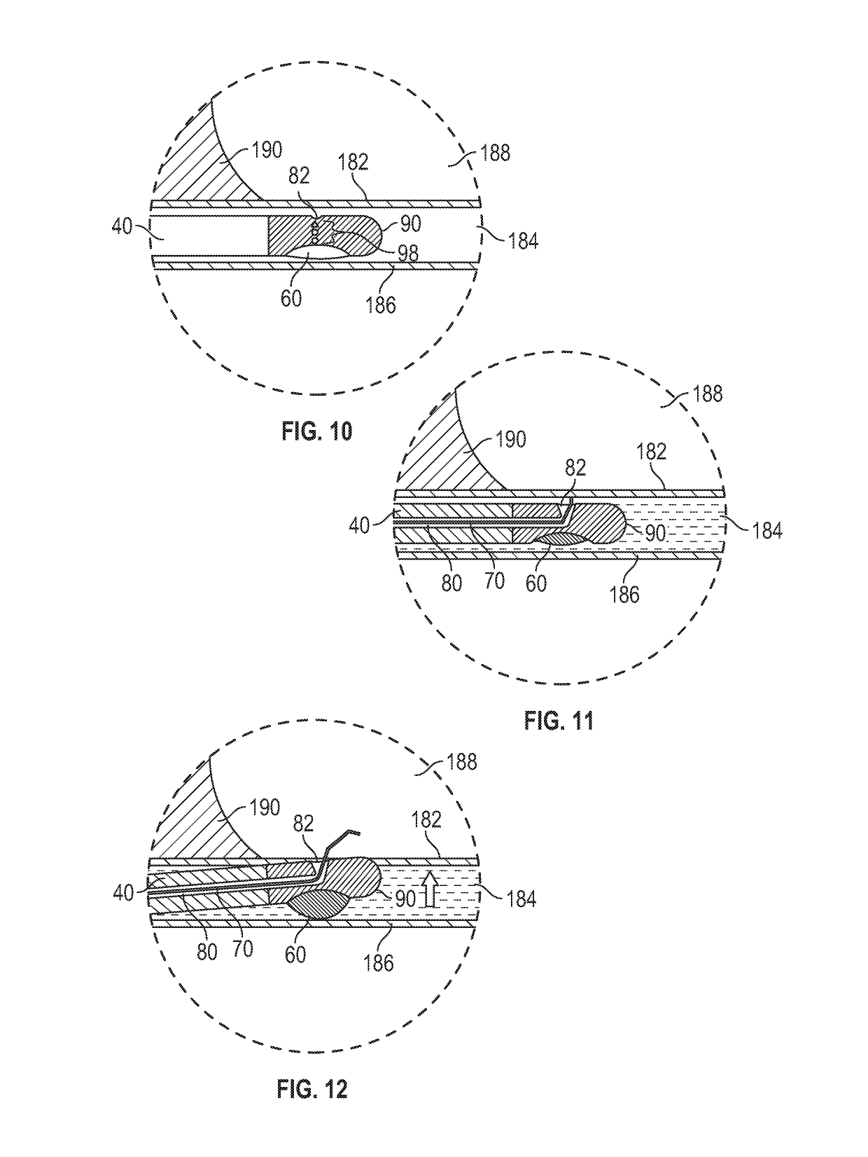

As shown in FIG. 10, once the distal end portion 90 reaches the distal side of the occlusion 190 and is within the subintimal space, an operation can determine the relative position and/or orientation of the distal end portion 90 and the true lumen 188 of the vessel 180. The operation can include a visualization technique, such as an antegrade angiogram. The markers 98 on the distal end portion 90 can be visualized, for example under fluoroscopy. As further shown in FIG. 11, the distal end portion 90 can be rotated so that the opening 82 faces toward the true lumen 188 of the vessel 180. Optionally, the distal end portion 90 can penetrate the distal cap of the occlusion 190 into the true lumen 188 of the vessel 180.

As shown in FIG. 11, the operator can advance a wire 70 inside the lumen 80 of the catheter 40. The wire 70 can be advanced toward, to, or through the opening 82. In cases where the distal end portion 90 is in the subintimal space, the operator can direct the wire 70 from inside the catheter 40 in the subintimal space into the true lumen 188 of the vessel 180.

As shown in FIG. 12, the balloon 60 or other expandable member of the distal end portion 90 can optionally be inflated or otherwise expanded to push the opening 282 closer to the true lumen 188 of the vessel 180. Inflation of the balloon 60 may allow the wire 70 to pierce through the intima layer 182 into the true lumen 188. Because the external adventitia layer 186 is more inelastic than the intima layer 182, the forces generated through inflation of the balloon 60 may cause the intima layer 182 to yield first, bending or folding towards the true lumen 188, rather than causing the external adventitia layer 186 to stretch.

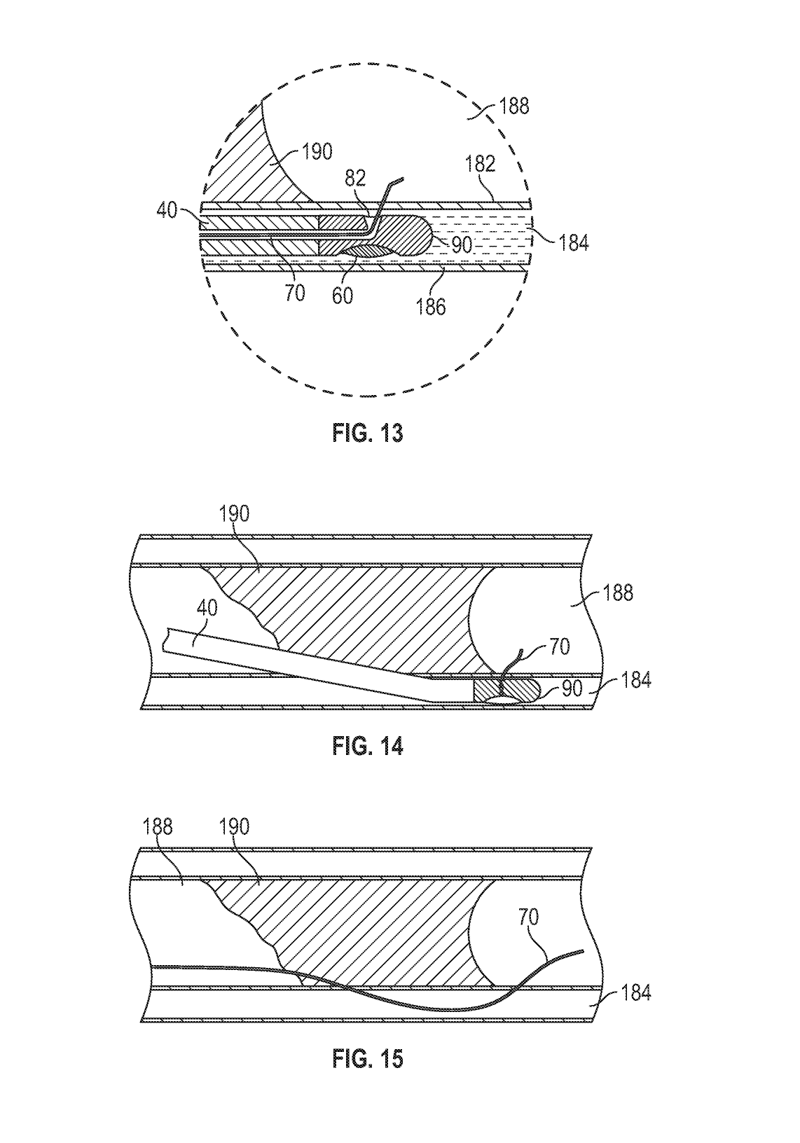

As shown in FIGS. 13 and 14, the balloon 60 can be deflated in preparation for removal of the catheter 40. Once the wire 70 is advanced into the true lumen 188, the catheter 40 can be advanced over the wire. Once the catheter 40 is placed within the true lumen 188, the operator can inject contrast after removing the wire to confirm intra luminal position. Once intra luminal position is confirmed, a wire 70 can be re-introduced into the distal vessel preparing the vessel 180 for a therapy. As shown in FIG. 15, the catheter 40 can be retracted over the wire 70, so that the catheter 40 is removed from the body while the wire 70 remains in a position that spans the occlusion 190, for example at least partially within the media layer 184.

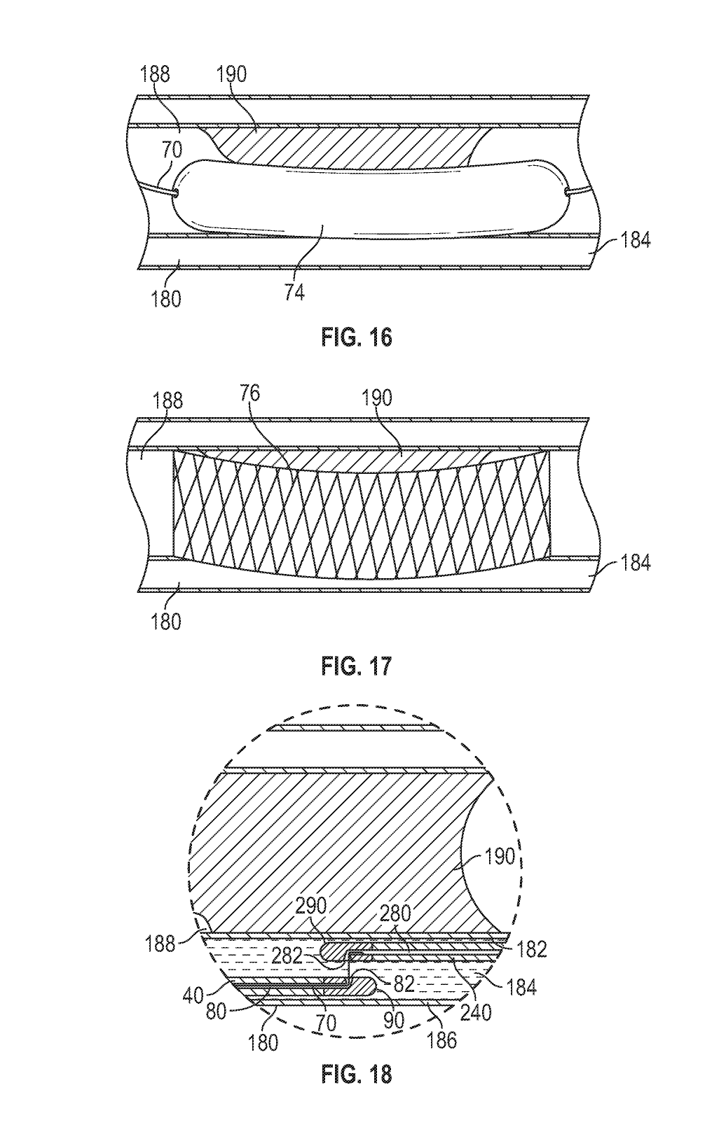

One or more of a variety of therapies can be facilitated by the position of the wire 70. As shown in FIG. 16, balloon angioplasty can be performed with a balloon device 74 after advancement over the wire 70. As shown in FIG. 18, a stent 76 can be deployed after being advanced over the wire 70. These and/or other therapies can help establish and maintain a fluid pathway through and/or around the occlusion 190. After these therapies, the wire 70 and/or other devices can be removed.

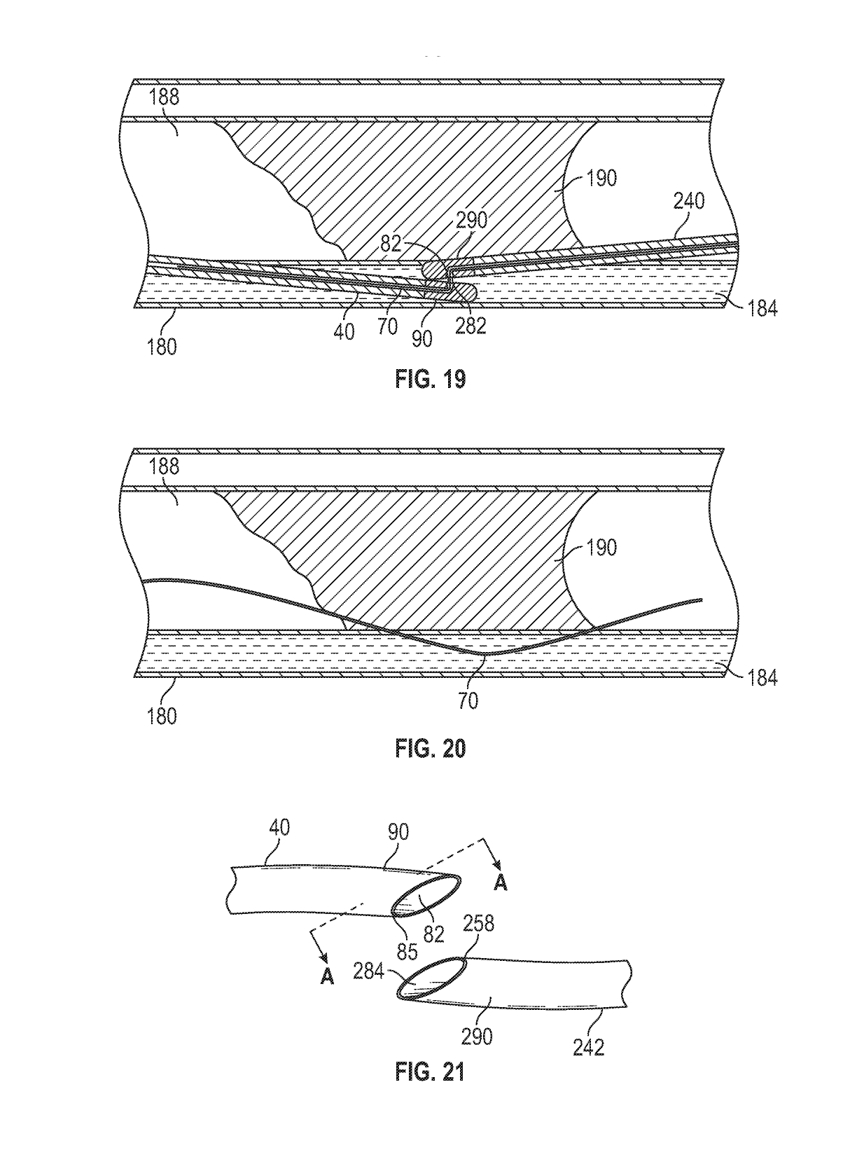

FIGS. 19-21 show a method involving two catheters 40 and 240. According to some embodiments, a modality of crossing the occlusion can be through bidirectional activation of the catheter 40. The antegrade catheter 40 can be provided substantially as illustrated in FIGS. 7-10. The antegrade catheter 40 can be activated until the catheter penetrates the occlusion 190 and possibly into the subintimal space. At this stage, another catheter 240 can be advanced from retrograde (e.g., popliteal/pedal) access by approaching from an opposite side of the occlusion 190 in the true lumen 188 of the vessel 180. For example, the distal end portion 290 of the retrograde catheter 240 can approach the occlusion from a distal end of the occlusion 190. The retrograde catheter 240 can be activated by rotation as described above with respect to the catheter 40. Once the retrograde catheter 240 penetrates the distal side of the occlusion 190, the catheter 240 can be advanced to the level of the antegrade catheter 40. At this stage, orientation of both catheters 40 and 240 can be performed as described above. Alignment can be performed so that the opening 82 of the antegrade catheter 40 is axially aligned with the opening 282 of the retrograde catheter 240. Furthermore, orientation can be performed so that the opening 82 of the antegrade catheter 40 is opposite and facing toward the opening 282 of the retrograde catheter 240. As shown in FIG. 18, the wire 70 from the antegrade catheter 40 can be advanced to the retrograde catheter 240. The wire 70 can also be advanced from the retrograde catheter 240. As discussed herein, the opening 282 of the retrograde catheter 240 can be different (e.g., larger, tapered, etc.) than the opening 82 of the antegrade catheter 40. For example, the opening 282 of the retrograde catheter 240 can be larger than the opening 82 of the antegrade catheter 40 to facilitate receipt of the wire 70 into the opening 282 and the lumen 280.

As shown in FIG. 19, once the wire 70 from the antegrade catheter 40 is advanced into the retrograde catheter, or vice versa, the wire 70 can be flossed. Flossing the wire 70 refers to the wire 70 being across the occlusion. As shown in FIG. 20, at this stage, the operator can proceed with revascularization as described above, including removal of the catheters 40 and 240.

It will be understood that the alignment and orientation of catheters (e.g., distal end portions and openings) can be achieved in a variety of locations. As shown in FIGS. 18 and 19, both the antegrade catheter 40 and the retrograde catheter 240 can be located in the same subintimal space on a same side of the true lumen 188 of the vessel 180. Additionally or alternatively, the antegrade catheter 40 and the retrograde catheter 240 can be located in different subintimal spaces on opposite sides of the true lumen 188 of the vessel 180, with the wire 70 extending there between on one or more sides of the occlusion 190 and/or through the occlusion 190. Additionally or alternatively, one of the antegrade catheter 40 and the retrograde catheter 240 can be located in a subintimal space and the other can be located within the true lumen 188 of the vessel 180. Additionally or alternatively, neither the antegrade catheter 40 nor the retrograde catheter 240 may be located in the subintimal space. Additionally or alternatively, neither the antegrade catheter 40 nor the retrograde catheter 240 may be located in the true lumen 188 of the vessel 180.

The antegrade catheter 40 and the retrograde catheter 240 may approach the occlusion 190 from opposite sides (e.g., proximal and distal) thereof within the true lumen 188 of the vessel 180. Accordingly, the wire or another device can span the occlusion 190 including across opposing sides (e.g., caps) thereof. Additionally or alternatively, the antegrade catheter 40 and the retrograde catheter 240 may approach the occlusion 190 from the same side (e.g., proximal or distal) within the true lumen 188 of the vessel 180. For example, the wire or another device can span a portion of the occlusion 190 and return to the same side (e.g., cap) of its approach. By further example, the wire or another device can extend about the occlusion 190 and return to the same side (e.g., cap) of its approach.

Alignment of openings for two separate catheters can be facilitated by magnetic couplings across the catheters. As shown in FIG. 21, an antegrade catheter 40 and a retrograde catheter 240 can each have a magnetic element. The opening 82 of the antegrade catheter 40 and the opening 282 of the retrograde catheter 240 can be oriented to connect when the magnetic elements are attracted to each other.

As shown in FIG. 21, the opening 82 forms a terminal end of the antegrade catheter 40 (e.g., at the distal end portion 90), and the opening 282 forms a terminal end of the retrograde catheter 240 (e.g., at the distal end portion 290). Each terminal end can include a flat or planar surface. The surface can form an angle with respect to the longitudinal axis of the corresponding catheter. The angle can be transverse (e.g., oblique) to the longitudinal axis, so that the terminal ends provide surfaces that can be coupled together when the catheters bend and contact each other. For example, the angle can be any non-zero angle (e.g., greater than 0 degrees, up to 90 degrees).

The first magnetic element 58 is within the distal end portion 90 at or near the terminal end thereof. The second magnetic element 258 is within the distal end portion 290 at or near the terminal end thereof. The first magnetic element 58 and/or the second magnetic element 258 can include a temporary magnet of a soft magnetic material or a permanent magnet of a hard magnetic material. As used herein, “magnet” can include a magnet of a hard magnetic material and/or a magnet of a soft magnetic material. Hard magnetic materials include materials that retain their magnetism even after the removal of an applied magnetic field. Magnets that include hard magnetic material can form permanent magnets. Hard magnetic materials include neodymium (NdFeB), iron-neodymium, iron-boron, cobalt-samarium, iron-chromium-cobalt, and combinations or alloys thereof. Soft magnetic materials include materials that are responsive to magnetic fields, but do not retain their magnetism after removal of an applied magnetic field. Magnets that include soft magnetic material can form temporary magnets. Soft magnetic materials include iron, iron-cobalt, iron-silicon, steel, stainless steel, iron-aluminum-silicon, nickel-iron, ferrites, and combinations or alloys thereof. It will be recognized that “hard magnetic” and “soft magnetic” does not necessarily relate to the rigidity of the materials. Additionally or alternatively, the first magnetic element 58 and/or the second magnetic element 258 can form an electromagnet. The electromagnet can be controllably activated by an operator when desired by providing an electrical current to the electromagnet. The magnitude of the magnetic attraction can be controlled by operation of the electromagnet.

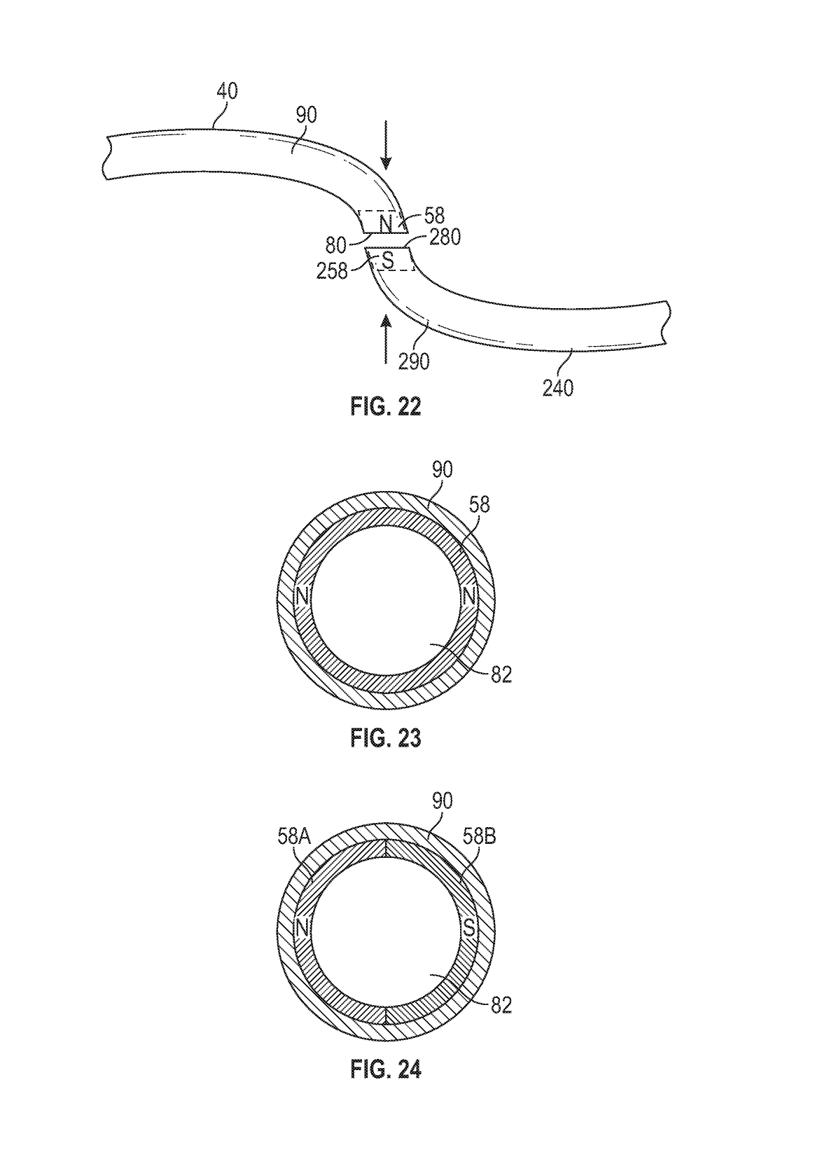

As shown FIG. 22, when the magnetic elements 58 and 258 are brought into proximity of each other and/or activated, the opening 82 of the antegrade catheter 40 and the opening 282 of the retrograde catheter 240 can be connected to form a continuous pathway. For example, attraction between the magnetic elements 58 and 258 can cause the antegrade catheter 40 and/or the retrograde catheter 240 to bend, flex, or deflect to meet each other. The attraction can urge the openings 82 and 282 to align so that the continuous pathway is formed. Furthermore, the terminal ends of the distal end portions 90 and 290 can form a seal or other resilient connection.

The magnetic elements 58 and 258 can be oriented to facilitate the magnetic attraction and coupling. For example, the polarities of the magnetic elements 58 and 258 can be aligned so that the opposing distal end portions 90 and 290 are magnetically attracted to each other.

Various magnet arrangements and configurations can be provided to achieve the functionality discussed herein. As shown in FIG. 23, one or more of the magnetic elements 58 and 258 can form an annular ring that surrounds the first opening. The annular ring can provide a magnetic field that extends through the corresponding opening. The annular ring can be embedded within, surrounded by, or surrounding the corresponding distal end portions.

As shown in FIG. 24, one or more of the magnetic elements 58 and 258 can include multiple magnets on opposing radial sides of a longitudinal axis. For example, separate magnets can extend along a radial side of the longitudinal axis. The separate magnets can have the same or different magnetic polarities. While only two magnets 58A and 58B are shown, it will be appreciated that any number of magnets can be provided with a circumferential distribution. For example, the end can include 2, 3, 4, 5, 6, 7, 8, 9, or more than 10 magnets. The magnets can have alternating polarities about the opening. The polarities of the magnets can correspond to opposing polarities in the magnets of an opposing catheter. Accordingly, the openings can be rotationally aligned with respect to each other based on the magnetic attraction of multiple individual magnets.

The magnetic attraction and alignment discussed herein can be applied to the other methods illustrated and discussed herein. For example, magnetic elements can be aligned and/or activated during an alignment of opening, such as that illustrated in FIG. 18. After alignment of openings with magnetic coupling, the wire can be passed through both openings to span both catheters.

Additionally or alternatively, other devices, such as a magnetic snare, can be used to magnetically couple to one or more of the catheters. The snare can be magnetic such that it magnetically couples to magnetic elements of a catheter. Additional operations, such as adjustments and/or retrieval can be performed with the magnetic snare.

The apparatuses and methods discussed herein are not limited to treatment of any particular vessels, but may include any number of different types of vessels. For example, in some aspects, vessels may include arteries or veins. The vessels may have bifurcations and/or sharp turns. In some aspects, the vessels may be suprathoracic vessels (e.g., vessels in the neck or above), intrathoracic vessels (e.g., vessels in the thorax), subthoracic vessels (e.g., vessels in the abdominal area or below), lateral thoracic vessels (e.g., vessels to the sides of the thorax such as vessels in the shoulder area and beyond), or other types of vessels and/or branches thereof.

In some aspects, the suprathoracic vessels may comprise at least one of intracranial vessels, cerebral arteries, and/or any branches thereof. For example, the suprathoracic vessels may comprise at least one of a common carotid artery, an internal carotid artery, an external carotid artery, a middle meningeal artery, superficial temporal arteries, an occipital artery, a lacrimal (ophthalmic) artery, an accessory meningeal artery, an anterior ethmoidal artery, a posterior ethmoidal artery, a maxillary artery, a posterior auricular artery, an ascending pharyngeal artery, a vertebral artery, a left middle meningeal artery, a posterior cerebral artery, a superior cerebellar artery, a basilar artery, a left internal acoustic (labyrinthine) artery, an anterior inferior cerebellar artery, a left ascending pharyngeal artery, a posterior inferior cerebellar artery, a deep cervical artery, a highest intercostal artery, a costocervical trunk, a subclavian artery, a middle cerebral artery, an anterior cerebral artery, an anterior communicating artery, an ophthalmic artery, a posterior communicating artery, a facial artery, a lingual artery, a superior laryngeal artery, a superior thyroid artery, an ascending cervical artery, an inferior thyroid artery, a thyrocervical trunk, an internal thoracic artery, and/or any branches thereof. The suprathoracic vessels may also comprise at least one of a medial orbitofrontal artery, a recurrent artery (of Heubner), medial and lateral lenticulostriate arteries, a lateral orbitofrontal artery, an ascending frontal (candelabra) artery, an anterior choroidal artery, pontine arteries, an internal acoustic (labyrinthine) artery, an anterior spinal artery, a posterior spinal artery, a posterior medial choroidal artery, a posterior lateral choroidal artery, and/or branches thereof. The suprathoracic vessels may also comprise at least one of perforating arteries, a hypothalamic artery, lenticulostriate arteries, a superior hypophyseal artery, an inferior hypophyseal artery, an anterior thalamostriate artery, a posterior thalamostriate artery, and/or branches thereof. The suprathoracic vessels may also comprise at least one of a precentral (pre-Rolandic) and central (Rolandic) arteries, anterior and posterior parietal arteries, an angular artery, temporal arteries (anterior, middle and posterior), a paracentral artery, a pericallosal artery, a callosomarginal artery, a frontopolar artery, a precuneal artery, a parietooccipital artery, a calcarine artery, an inferior vermian artery, and/or branches thereof.

In some aspects, the suprathoracic vessels may also comprise at least one of diploic veins, an emissary vein, a cerebral vein, a middle meningeal vein, superficial temporal veins, a frontal diploic vein, an anterior temporal diploic vein, a parietal emissary vein, a posterior temporal diploic vein, an occipital emissary vein, an occipital diploic vein, a mastoid emissary vein, a superior cerebral vein, efferent hypophyseal veins, infundibulum (pituitary stalk) and long hypophyseal portal veins, and/or branches thereof.

The intrathoracic vessels may comprise the aorta or branches thereof. For example, the intrathoracic vessels may comprise at least one of an ascending aorta, a descending aorta, an arch of the aorta, and/or branches thereof. The descending aorta may comprise at least one of a thoracic aorta, an abdominal aorta, and/or any branches thereof. The intrathoracic vessels may also comprise at least one of a subclavian artery, an internal thoracic artery, a pericardiacophrenic artery, a right pulmonary artery, a right coronary artery, a brachiocephalic trunk, a pulmonary trunk, a left pulmonary artery, an anterior interventricular artery, and/or branches thereof. The intrathoracic vessels may also comprise at least one of an inferior thyroid artery, a thyrocervical trunk, a vertebral artery, a right bronchial artery, a superior left bronchial artery, an inferior left bronchial artery, aortic esophageal arteries, and/or branches thereof.

In some aspects, the intrathoracic vessels may also comprise at least one of a right internal jugular vein, a right brachiocephalic vein, a subclavian vein, an internal thoracic vein, a pericardiacophrenic vein, a superior vena cava, a right superior pulmonary vein, a left brachiocephalic vein, a left internal jugular vein, a left superior pulmonary vein, an inferior thyroid vein, an external jugular vein, a vertebral vein, a right highest intercostal vein, a 6th right intercostal vein, an azygos vein, an inferior vena cava, a left highest intercostal vein, an accessory hemiazygos vein, a hemiazygos vein, and/or branches thereof.

In some aspects, the subthoracic vessels may comprise at least one of renal arteries, inferior phrenic arteries, a celiac trunk with common hepatic, left gastric and splenic arteries, superior suprarenal arteries, a middle suprarenal artery, an inferior suprarenal artery, a right renal artery, a subcostal artery, 1st to 4th right lumbar arteries, common iliac arteries, an iliolumbar artery, an internal iliac artery, lateral sacral arteries, an external iliac artery, a testicular (ovarian) artery, an ascending branch of deep circumclex iliac artery, a superficial circumflex iliac artery, an inferior epigastric artery, a superficial epigastric artery, a femoral artery, a ductus deferens and testicular artery, a superficial external pudendal artery, a deep external pudendal artery, and/or branches thereof. The subthoracic vessels may also comprise at least one of a superior mesenteric artery, a left renal artery, an abdominal aorta, an inferior mesenteric artery, colic arteries, sigmoid arteries, a superior rectal artery, 5th lumbar arteries, a middle sacral artery, a superior gluteal artery, umbilical and superior vesical arteries, an obturator artery, an inferior vesical and artery to ductus deferens, a middle rectal artery, an internal pudendal artery, an inferior gluteal artery, a cremasteric, pubic (obturator anastomotic) branches of inferior epigastric artery, a left colic artery, rectal arteries, and/or branches thereof.

In some aspects, the lateral thoracic vessels may comprise at least one of humeral arteries, a transverse cervical artery, a suprascapular artery, a dorsal scapular artery, and/or branches thereof. The lateral thoracic vessels may also comprise at least one of an anterior circumflex humeral artery, a posterior circumflex humeral artery, a subscapular artery, a circumflex scapular artery, a brachial artery, a thoracodorsal artery, a lateral thoracic artery, an inferior thyroid artery, a thyrocervical trunk, a subclavian artery, a superior thoracic artery, a thoracoacromial artery, and/or branches thereof.

The apparatus and methods discussed herein are not limited to the deployment and use of a balloon or stent within the vascular system but may include any number of further treatment applications. Other treatment sites may include areas or regions of the body such as organ bodies.

Various examples of aspects of the disclosure are described below as clauses for convenience. These are provided as examples, and do not limit the subject technology.

Clause A. A catheter comprising: a distal end portion; a lumen extending longitudinally within the catheter and radially to an opening in a first radial side at the distal end portion of the catheter; and an inflatable balloon on a second radial side at the distal end portion of the catheter, opposite the first radial side, the inflatable balloon configured to expand radially away from the lumen when expanded.

Clause B. A system, comprising: a first catheter comprising: a first lumen extending longitudinally within the first catheter to an opening in a first end portion of the first catheter; and a first magnetic element at the first end portion; and a second catheter comprising: a second lumen extending longitudinally within the second catheter to an opening in a second end portion of the second catheter; and a second magnetic element at the second end portion, wherein the first magnetic element and the second magnetic element are configured to attract each other and align the first opening with the second opening.

Clause C. A method comprising: positioning a first catheter on a first side of an occlusion within a body vessel; advancing a first end portion of the first catheter into a subintimal tissue adjacent to the occlusion; positioning a second catheter on a second side of the occlusion; advancing a second end portion of the second catheter into the subintimal tissue; and advancing a wire through a lumen of the first catheter, out of a first opening of the first catheter, and into a second opening of the second catheter while the first opening is facing the second opening.

Clause D. A method comprising: positioning a catheter in a region of a body vessel on a first side of an occlusion; advancing an end portion of the catheter into a subintimal tissue adjacent to the occlusion and to a region of the subintimal tissue adjacent to a region of the body vessel on a second side of the occlusion; positioning an opening in a first radial side of the end portion to face body vessel; inflating a balloon on a second radial side of the end portion, opposite the first radial side, such that the end portion is deflected toward the body vessel; and advancing a wire out of the opening and into the body vessel while the end portion is within the subintimal tissue.

Clause E. A catheter comprising: a distal end portion; a lumen extending longitudinally within the catheter and radially to a port in a radial side at the distal end portion of the catheter; and at least three radiopaque markers axially aligned with the port, wherein one of the radiopaque markers has an a radially asymmetric shape.

In one or more aspects, the catheters, systems, and/or method of any preceding paragraph, either alone or in combination, can further include one or more features of the additional clauses described below.

Element 1. the catheter is a first catheter, the distal end portion is a first distal end portion, the lumen is a first lumen, and the opening is a first opening; and a second catheter comprising: a second distal end portion; and a second lumen extending longitudinally within the second catheter and radially to a second opening in a third radial side at the second distal end portion of the second catheter, wherein the second opening is larger than the first opening.

Element 2. the balloon is axially aligned with the opening.

Element 3. at least three radiopaque markers axially aligned with the opening, wherein one of the radiopaque markers has a radially asymmetric shape.

Element 4. the radially asymmetric shape is a triangle.

an end of the radially asymmetric shape points radially toward the opening.

Element 5. at least two of the radiopaque markers have different shapes.

Element 6. at least two of the radiopaque markers have the same shape.

Element 7. the first magnetic element forms an annular ring that surrounds the first opening, and the second magnetic element forms an annular ring that surrounds the second opening.

Element 8. the first opening forms a first terminal end of the first catheter that is transverse to a longitudinal axis of the first catheter, and the second opening forms a second terminal end of the second catheter that is transverse to a longitudinal axis of the second catheter.

Element 9. the first magnetic element comprises multiple magnets on opposite radial sides of a longitudinal axis of the first catheter, and the second magnetic element comprises multiple magnets on opposite radial sides of a longitudinal axis of the second catheter.

Element 10. each of the first magnetic element and the second magnetic element comprises an electromagnet.

Element 11. the advancing comprises advancing the wire until a first portion of the wire is in the body vessel on a first side of the occlusion, a second portion of the wire is within the subintimal tissue, and a third portion of the wire is in the body vessel on the second side of the occlusion.

Element 12. removing the first catheter and the second catheter from the subintimal tissue without removing the wire from the subintimal tissue.

Element 13. advancing a device along the wire from a first side of the occlusion, through the subintimal tissue, to a second side of the occlusion.

Element 14. providing a channel through the subintimal tissue for fluid flow between the first side of the occlusion and the second side of the occlusion.

Element 15. advancing the second end portion comprises aligning the first opening of the first end portion with the second opening of the second end portion.

Element 16. the aligning comprises allowing a first magnetic element of the first end portion to attract to a second magnetic element of the second end portion.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A catheter comprising:
a distal end portion;
a lumen extending longitudinally within the catheter and radially to an opening in a first radial side at the distal end portion of the catheter; and
an inflatable balloon on a second radial side at the distal end portion of the catheter, opposite the first radial side, the inflatable balloon configured to expand radially away from the lumen when expanded.

2. The catheter of claim 1, wherein the balloon is axially aligned with the opening.

3. The catheter of claim 1, further comprising at least three radiopaque markers axially aligned with the opening, wherein one of the radiopaque markers has a radially asymmetric shape.

4. The catheter of claim 3, wherein the radially asymmetric shape is a triangle.

5. The catheter of claim 3, wherein an end of the radially asymmetric shape points radially toward the opening.

6. The catheter of claim 3, wherein at least two of the radiopaque markers have different shapes.

7. The catheter of claim 3, wherein at least two of the radiopaque markers have the same shape.

8. A system, comprising:
the catheter of claim 1, wherein the catheter is a first catheter, the distal end portion is a first distal end portion, the lumen is a first lumen, and the opening is a first opening; and
a second catheter comprising:
a second distal end portion; and
a second lumen extending longitudinally within the second catheter and radially to a second opening in a third radial side at the second distal end portion of the second catheter, wherein the second opening is larger than the first opening.

9. A system, comprising:
a first catheter comprising:
a first lumen extending longitudinally within the first catheter to an opening in a first end portion of the first catheter; and
a first magnetic element at the first end portion; and
a second catheter comprising:
a second lumen extending longitudinally within the second catheter to an opening in a second end portion of the second catheter; and
a second magnetic element at the second end portion, wherein the first magnetic element and the second magnetic element are configured to attract each other and align the first opening with the second opening.

10. The system of claim 9, wherein the first magnetic element forms an annular ring that surrounds the first opening, and the second magnetic element forms an annular ring that surrounds the second opening.

11. The system of claim 9, wherein the first opening forms a first terminal end of the first catheter that is transverse to a longitudinal axis of the first catheter, and the second opening forms a second terminal end of the second catheter that is transverse to a longitudinal axis of the second catheter.

12. The system of claim 9, wherein the first magnetic element comprises multiple magnets on opposite radial sides of a longitudinal axis of the first catheter, and the second magnetic element comprises multiple magnets on opposite radial sides of a longitudinal axis of the second catheter.

13. The system of claim 9, wherein each of the first magnetic element and the second magnetic element comprises an electromagnet.

14. A method comprising:
positioning a first catheter on a first side of an occlusion within a body vessel;
advancing a first end portion of the first catheter into a subintimal tissue adjacent to the occlusion;
positioning a second catheter on a second side of the occlusion;
advancing a second end portion of the second catheter into the subintimal tissue; and
advancing a wire through a lumen of the first catheter, out of a first opening of the first catheter, and into a second opening of the second catheter while the first opening is facing the second opening.

15. The method of claim 14, wherein the advancing comprises advancing the wire until a first portion of the wire is in the body vessel on a first side of the occlusion, a second portion of the wire is within the subintimal tissue, and a third portion of the wire is in the body vessel on the second side of the occlusion.

16. The method of claim 14, further comprising removing the first catheter and the second catheter from the subintimal tissue without removing the wire from the subintimal tissue.

17. The method of claim 14, further comprising advancing a device along the wire from a first side of the occlusion, through the subintimal tissue, to a second side of the occlusion.

18. The method of claim 17, further comprising providing a channel through the subintimal tissue for fluid flow between the first side of the occlusion and the second side of the occlusion.

19. The method of claim 14, wherein advancing the second end portion comprises aligning the first opening of the first end portion with the second opening of the second end portion.

20. The method of claim 19, wherein the aligning comprises allowing a first magnetic element of the first end portion to attract to a second magnetic element of the second end portion.
